(19) United States
(12) Patent Application Publication
(10) Pub. No.: US 2021/0228752 A1
(43) Pub. Date: Jul. 29, 2021

(54) Method and Device for Cleaning and Disinfecting Objects

(71) Applicant: Sing-Hong Stefan Chang, Aachen (DE)

(72) Inventor: Sing-Hong Stefan Chang, Aachen (DE)

(21) Appl. No.: 17/257,026

(22) PCT Filed: Jun. 10, 2019
(86) PCT No.: PCT/DE2019/100528

§ 371 Date: Dec. 30, 2020

(30) Foreign Application Priority Data
Jul. 4, 2018 (DE) 20 2018 103 840.0
Jun. 10, 2019 (DE) 20 2019 103 254.5

(51) Int. Cl. A61L 2/20; A61L 2/06; A61L 2/10; A61L 2/238

(52) U.S. Cl. CPC A61L 2/202; A61L 2/06; A61L 2/10; A61L 2202/122; A61L 2202/26; A61L 2202/11; A61L 2/238

(57) ABSTRACT

The invention relates to a method and a device for cleaning and disinfecting at least one object, wherein the device has a chamber surrounded by a housing, in which the at least one object is accommodated on at least one receiving element. The device has at least three of the following means A) to D): A) at least one UV lamp arranged in the chamber on the receiving element for generating UV-C light in the non-visible wavelength range, B) at least one means for generating ozone, C) at least one device for setting and/or changing the temperature in the chamber and D) at least one device for setting and/or changing the pressure in the chamber, an air inlet pointing from outside the housing into the interior of the chamber and having an air inlet and an air outlet pointing from the chamber out of the housing.

### CROSS REFERENCE TO RELATED APPLICATIONS

This application is the U.S. national stage of International Application No. PCT/DE2019/100528, filed on Jun. 10, 2019. The international application claims the priority of DE 202018103840.0 filed on Jul. 4, 2018 and the priority of DE 202019103254.5 filed on Jun. 10, 2019; all applications are incorporated by reference herein in their entirety.

### BACKGROUND

The invention relates to a method and a device for cleaning and disinfecting objects according to the preamble of claim 1 and claim 13 and is used in particular for cleaning and disinfecting objects including textiles, for example clothing, shoes, sports equipment, backpacks, or helmets and the like and thus in particular objects or pieces of clothing which are preferably worn on the body and furthermore blankets, mattresses, carpets, and also stuffed animals or also textile upholstery in or from vehicles.

Such objects or textiles are preferably to be cleaned by means of a washing machine and subsequent drying in a clothes dryer, wherein soiling, germs, bacteria, and fungi are removed. In nature, bacteria and fungi feed on the sweat and dead skin particles released by humans. A gas is excreted as a digestion product, which is perceptible as an unpleasant smell. However, not all products coming into contact with sweat or other odorous substances (smoke or food) may be cleaned in the washing machine, or the material suffers from too many washing processes. For example, shoes, in particular sports shoes, gloves (in particular if they include leather), helmets, and the like should not or cannot be washed in a washing machine or not even completely by hand.

Bacteria in liquids and materials can be killed and many organisms can be inactivated or destroyed by irradiation using ultraviolet light. This can be considered to be disinfection of the irradiated region. These microscopic organisms comprise, for example, spore-forming and non-spore-forming forms of life or viruses or bacteriophages or cysts.

A method for disinfecting liquids and gases and also devices for use of the mentioned method are known from document DE 698 01 450 T2. The document relates in particular to methods for disinfecting liquids and gases by light, which is radiated by light guides into the liquids and gases. The light is used in the form of ultraviolet radiation (UVA, UVB, UVC), since this radiation is particularly suitable for killing bacteria or microscopic harmful microorganisms. The light can moreover originate from the visible range of the spectrum. The method comprises the distribution of at least one light guide in a region which contains the liquids or gases to be disinfected. At least one radiation unit having a light source of high intensity is oriented and the liquid or the gas is subsequently irradiated over a predetermined time period. The radiation unit is designed in the form of a laser.

A cabinet for cleaning clothing is described in document EP 2 273 004 B1, wherein the cabinet has a cleaning chamber for the clothing to be cleaned and means for generating ozone from air. The ozone is introduced into the cleaning chamber and brought into contact with the clothing. The cabinet has a device chamber, in which ozone for the cleaning of the clothing is generated and supplied to the clothing. The device chamber is formed separated by a wall from the cleaning chamber.

A device for sterilizing shoes is known from document U.S. Pat. No. 4,981,651 A. The device has an oblong lamp for emitting ultraviolet radiation in a housing and also a heating element and a fan. The device can be introduced into a shoe, wherein the shoe sole is sterilized by means of the lamp and the heating element. The growth of fungi and bacteria is stopped. However, only air circulation within the UV radiation source, but not in the chamber located around it is described. An exhaust unit and a filter are not disclosed.

A similar device is described in US 2016/0339126 A1 (D1). UV sources are arranged in a housing therein, which are at least partially introduced into textiles to be cleaned. An exhaust unit is not provided here.

A chamber for treating textiles having an air supply, a heating source, and a UV radiation source, using which ozone is generated, is disclosed in U.S. Pat. No. 3,877,152 A. A vacuum can be applied to the chamber.

Document CH 359 113 A also describes a chamber to which a vacuum is applied and thus an airflow is generated. The vacuum is used to discharge the ozone and the dirt particles released from the laundry.

A method and a device for cleaning textiles are known from document DE 10 2012 209 823 A1, wherein these textiles are introduced through an opening into the chamber of a domestic appliance. Air is introduced into the chamber of the dryer. The air is irradiated using UV-C light, 100 to 280 nm, wherein an ozone generator provided for this purpose is arranged outside the chamber. Efficient cleaning of the inside regions of the pieces of clothing or the surface of other materials or objects is not possible using this solution.

Document DE 10 2007 037 984 A discloses a device having an air circulation and an ozone source which can be introduced into the textiles. The air can flow through the fabric. Ozone and dirt particles are removed by a separating device. The UV source is located at the chamber-side end of the air supply, wherein the airflow is not divided for multiple UV radiation sources, however. Furthermore, a pressure change is not provided.

A device for drying shoes is known from document US 2009/0193676 A1, which separates the ozone generation and the UV radiation from one another, however.

Efficient cleaning and disinfecting of the pieces of clothing or the surface of other materials or objects such as blankets, mattresses, carpets, and also stuffed animals or also textile upholstery in or from vehicles is not possible using the above-mentioned solutions.

Furthermore, ozone is already harmful to the health in much lower concentrations in the respiratory air, in particular the ozone stress causes irritation of the respiratory tract.

A device for sterilizing human shoes is also described in document DE 11 2007 000 615 T5, which has a light source which emits radiation in a wavelength range that sterilizes the shoe,

in that the growth of microorganisms which are present in an inner region of the shoe is inhibited or these are destroyed; a carrier for the light source in order to place it in a position in order to orient the radiation on the inner region of the shoe; and a light blocking unit, which, during the sterilization, prevents the radiation from harming a person who is located close to the shoe. Furthermore, this solution relates to a device for sterilizing human shoes having an opening into which the foot of a person is inserted in order to stand on the shoe, comprising: a light source, which emits radiation in a visible wavelength range that sterilizes the shoe in that the growth of microorganisms which are present in an inner region of the shoe is inhibited; and a carrier for the light source in order to place it in a position in order to orient the radiation into the inner region of the shoe in order to sterilize it.

Both of these above-mentioned solutions have the disadvantage that adhering soiling is not removed.

### SUMMARY

The object of the invention is to provide a method and a device for cleaning and disinfecting at least one object, in particular at least one recess or at least one cavity in an object, wherein environmentally-friendly antibacterial cleaning is enabled without the application of chemical additives and the device has a simply designed structure. This object is achieved by the characterizing features of the independent claims. Advantageous embodiments result from the dependent claims.

### DETAILED DESCRIPTION

According to the method, the cleaning and disinfecting of at least one object, in particular a piece of clothing and/or at least one recess or one cavity in the object, is carried out in that the object is received through an opening in a chamber enclosed by a housing at a receptacle element, wherein at least three of the following techniques a) to d) are implemented during a cleaning cycle in the chamber in an intelligent manner:
a) UVC light in the nonvisible wavelength range is generated by means of at least one UVC lamp,
b) ozone is generated by at least one means for ozone generation,
c) the temperature modes are settable and/or selectable,
d) the pressure is settable and/or changeable.

By means of an air inlet leading from the housing into the chamber and an air outlet leading from the chamber out of the housing, an airflow is generated inside the chamber, which is intentionally conducted over the UV lamp and is guided on or in the object to be cleaned, in particular to be disinfected.

In this case, objects are preferably cleaned and disinfected which consist of textile material or include textile material, which can also be combined with other materials such as plastic, leather, etc.

The at least three techniques which can be combined as desired from the above-mentioned techniques a) to e) ensure a good cleaning result and significantly reduce the contamination with bacteria and germs/microorganisms.

The at least three techniques used are preferably carried out simultaneously during a cleaning cycle. However, it is also possible to carry out the selected techniques in succession in any sequence or also alternately.

A UV-C lamp is preferably used as the means for ozone generation, using which ozone is generated by the emission of UV-C light, because ions are formed by the UV-C radiation which cause a conversion of air oxygen into ozone. The ozone is extremely reactive and has a strong germ-killing effect. The UV-C lamp is preferably dimmable (for example by an upstream or integrated dimmer), so that the radiation intensity of the UV-C lamp can be changed as a function of the degree of soiling of the objects to be cleaned. For a high degree of soiling, a high intensity of the UV-C lamp and therefore more ozone is generated, for a low degree of soiling, a lower intensity of the UV-C lamp is preferred and thus less ozone is generated. The wavelength of the UV-C lamps is between 100 nm and 800 nm, preferably between 100 nm and 300 nm, particularly preferably between 150 nm and 280 nm.

The temperature modes of the device are in particular settable or changeable in such a way that the temperature in the chamber rises from a low temperature to a higher temperature or is reduced from a high temperature to a lower temperature or the temperature alternately rises and sinks again or sinks and rises again. The temperature can be settable and/or changeable in this case in a temperature range from −50° C. to 100° C., preferably in a temperature range from −30° C. to 60° C., particularly preferably in a temperature range between −10° C. and 50° C.

In particular stubborn adhering materials are loosened by the temperature differences, so that they can be released more easily.

Furthermore, a negative pressure and/or an overpressure can be generated in the chamber in relation to the atmospheric pressure. The pressure in the chamber can rise from a low pressure to a higher pressure or can be reduced from a high pressure to a low pressure. It is also possible that the pressure in the chamber alternately rises and sinks again or sinks and rises again.

The pressure in the chamber can be settable and/or changeable in a pressure range between 0.001 bar to 10 bar, in particular between 0.1 bar and 2 bar. To achieve such a pressure difference, the chamber can be terminated airtight within the housing.

Dirt and other adhering materials are released from the object to be cleaned by the pressure differences.

It is furthermore possible that silver nanoparticles are introduced from a reservoir into the chamber, these can have a particle size from 1 nm to 1000 nm.

Is possible to introduce the silver particles into the chamber only after completion of the other techniques, so that they are laid over the surface of the object and thus form a future protection, since they have antibacterial properties or have a good bactericidal activity.

In a cleaning cycle, heated or cooled air is advantageously introduced into the chamber and irradiated in the chamber using ultraviolet light from at least one UV-C light source. By means of the UV-C light source, ozone is generated in the chamber and thus disinfection and cleaning of the at least one object is implemented, wherein the UV-C light source is at least partially introduced into the recesses or the cavity of the object. An alternating pressure is generated by means of a suction device in the chamber, whereby soiling of the object is released and the dirt particles are transported away.

In one advantageous embodiment, the airflow can be heated by means of a heating element in the region of the suction. The heating element is preferably integrated into the duct in the form of a heating fan.

Water steam is particularly preferably admixed to the airflow in the region of the supply into the chamber, whereby a smoothing effect is exerted on the textiles. Ironing the pieces of clothing can then be omitted.

The water steam is produced in a water tank having integrated heating element. An aroma is preferably added to the airflow and/or the chamber. The aroma elements can be replaceable and can be available in various aromas.

Extremely reliable cleaning and disinfecting of the object is thus achieved. In particular, recesses and cavities present in the object or clothing can be cleaned and disinfected if the UV lamp is at least partially introduced therein. Because the pressure differences also act in the cavities and the temperature in the chamber is cooled or heated (possibly also alternating), cavities and recesses of pieces of clothing (for example the interior of shoes or gloves) or also of bags, backpacks, helmets, crash helmets, caps, baseball caps, and other equipment are cleaned and disinfected. It is particularly advantageous if these objects consist of textile material or include this and cannot be washed or can only be washed in a restricted way.

The device for cleaning and disinfecting at least one object, in particular a piece of clothing or at least one recess or cavity in an object, includes a chamber enclosed by a housing, in which the object is hung on a receptacle element, wherein the device includes at least three of the following means A) to D) according to the invention:
A) at least one UV lamp arranged in the chamber on the receptacle element for generating UV-C light in the nonvisible wavelength range,
B) at least one means for ozone generation,
C) at least one unit for setting and/or changing the temperature in the chamber,
D) at least one unit for setting and/or changing the pressure in the chamber.

Furthermore, the device includes an air supply pointing from outside the housing into the interior of the chamber having an air inlet and at least one heating element extending along the air supply and an air outlet pointing from the chamber out of the housing.

A fan, which suctions in or exhausts air, respectively, is preferably arranged on the air inlet and the air outlet.

The device can include, for example, all of these means A) to D), wherein using these means at least three of the technologies which can thus be carried out are implemented during a cleaning cycle as needed.

At least one UV lamp for generating (nonvisible) UV-C radiation and for generating ozone is advantageously arranged on the receptacle element in the form of a hanger arranged in the chamber. Furthermore, the device includes the exhaust unit for generating a negative pressure in the chamber. The dirt particles released from the object are also exhausted using this exhaust unit.

At least two UV lamps are advantageously arranged on the receptacle element adjacent to one another and spaced apart from one another, wherein the UV lamps are dimmable in particular.

The receptacle element is designed to be adjustable in its width in one advantageous embodiment. This is preferably carried out by means of carrier elements movable outward, which are adjustable continuously or by means of detents.

The receptacle element is fastened removably and/or replaceably in the chamber. The object can thus conveniently be fixed on the receptacle element outside the chamber and subsequently hung in the chamber.

The cleaning cycle is advantageously settable by means of a control unit and the parameters such as temperature and/or pressure and/or feed of fragrances and/or silver nanoparticles are settable.

In one advantageous embodiment, an aroma element is arranged in the air supply. The aroma element can be settable in such a way that the desired intensity of the aroma of the clothing or the textile is set.

The device preferably includes a water tank having a heating element in such a way that water steam can be generated and that the water steam can be supplied via a duct of the air supply. A combined airflow made up of heated air and water steam is thus introduced into the chamber.

Depending on the space conditions, the chamber can be able to be withdrawn from the housing by means of a rail system or can include a frontal door.

The method according to the invention is used in particular for cleaning and disinfecting textiles, in particular cavities formed by textiles and textile planar formations, wherein the textiles (or at least one textile) is introduced through an opening into a chamber formed by a housing. In a first method step, temperature-variable air, preferably heated or cooled air, is preferably introduced into the chamber and subsequently the textile(s) is/are irradiated using ultraviolet light from at least one UV source, wherein ozone is generated in the chamber by means of the UV source and disinfecting and cleaning of the textiles (objects) takes place. The cavity formed by at least one textile is arranged on at least one UV source in such a way that the UV source can be at least partially introduced into the textiles or can be positioned near the textile. The germs and bacteria are killed by the combination of UV radiation and the ozone formed, whereby a disinfection of the relevant textile is implementable. The cleaning effect is improved by the temperature and the pressure in the chamber.

The generated ozone is removed through a filter introduced in the region of the exhaust unit when the cleaned air is exhausted or expelled from the chamber by means of the exhaust unit.

A negative pressure down to a vacuum is alternately generated in the chamber by means of the exhaust unit, by means of which soiling in the textiles is released and released dirt particles are transported away by means of the exhaust.

The UV source operates at a wavelength of the ultraviolet radiation in the nonvisible UV-C range, wherein the wavelength is between 100 nm and 800 nm, preferably between 100 nm and 300 nm, particularly preferably between 150 nm and 280 nm. The use of infrared radiation is also conceivable.

Air enriched with fragrances can preferably be supplied to the chamber. The textile to be cleaned or the object can thus be provided with a pleasant odor.

The device is used for cleaning textiles, in particular textiles afflicted with germs, bacteria, and fungi, wherein the device includes a housing and a chamber arranged in the interior of the housing having a receptacle element for textiles or objects. The device includes an air supply pointing from outside the housing into the interior of the chamber having at least one heating and/or cooling element extending along the air supply. At least one UV lamp is arranged on the receptacle element, wherein the UV lamp is provided for generating UV radiation in such a way that ozone can be generated in the chamber. Furthermore, the device includes an exhaust unit for generating a negative pressure in the chamber, whereby the air and dirt particles can be exhausted or conveyed out of the chamber, wherein the generated/used ozone is removed by a filter in such a way that filtered air is expelled.

In one advantageous embodiment, the exhaust unit is dimensioned in such a way that it can generate a vacuum in the chamber. Dirt and germs may thus be released from the textiles.

A supply for silver nanoparticles and/or fragrances is preferably provided on the device. The silver nanoparticles are used to avoid bacteria growth.

In this case, the silver nanoparticles and/or fragrances can be introduced into the chamber. Preferably, the silver nanoparticles and/or fragrances are introduced directly into the cavity/cavities formed by the textile(s). This preferably takes place via the air supply.

The air which can be introduced into the chamber can be heated by means of a heating coil, which is arranged around the air supply, or cooled via a cooling unit. A fan, which suctions in the air and conveys it into the chamber, is preferably arranged at the housing-side end of the air supply. A combined heating fan can also be used.

The exhaust is carried out in a similar way, wherein a second fan exhausts the air from the chamber and discharges it into the surroundings.

The cleaning cycle is preferably settable by means of a control unit, wherein individual or all parameters are adaptable. The parameters comprise the temperature and/or the pressure within the chamber and, if desired, the feed of fragrances and/or the silver nanoparticles. The chamber can furthermore be used for drying the textiles. The device uses the spectrum of the UV-C light as the UV source, wherein the combination of UV radiation, ozone, temperature programs, and vacuum-air exchange usage eliminate bacteria and fungi. The parameters can be ascertained by the device independently via the input of properties of the object to be cleaned. These properties comprise, for example, the type of the material, degree of soiling, degree of moisture, or the like. The settable temperature programs comprise the option of heated or also strongly cooled air, which is conducted to the cleaning location to be applied. The ozone is synthesized directly at the UV-C source, the UV lamp, i.e., synthesized and applied at the desired region of application.

The vacuum air exchange system removes dead, living, and eliminated organisms from the textiles.

The chamber has an antibacterial and antimycotic effect during the cleaning cycle and in particular cleans the interior of the products. The disinfection and cleaning takes place without the use of chemicals, whereby a high level of environmental friendliness and material protection is achieved. Due to the use of water steam, only a small amount of water is also required for the cleaning. The interior of shoes, helmets, gloves, sporting equipment, T-shirts, shirts, suits (pieces of clothing), or bags are preferably cleaned using the chamber, since these textiles are only suitable to a limited extent or not at all for the washing machine and subsequent drying.

The device and the chamber arranged therein are designed in such a way that textiles can pass therein without problems and free of wrinkles and can be arranged or hung therein. The dimensions with respect to width, height, and depth are preferably 30/120/60 cm. An enlargement of the dimensions has no influence on the effectivity of the mode of action. The device is preferably not to fall below the cited dimensions.

The device according to the invention is antibacterial and acts against various types of mites such as house mites and itch mites and also against fungi in the form of foot fungus or skin fungus.

Because heated/cooled air is conducted in the above-mentioned temperature range into the chamber by means of the air supply and the UV-C radiation source is positioned at the chamber-side end of the air supply and the UV-C radiation sources are introduced into the textiles or are positioned close to them, a better penetration of the textiles with the ozone via the airflow acting directly thereon is ensured than in the prior art and a better cleaning effect or disinfection effect is thus achieved.

This effect is improved still further if the receptacles for the objects to be cleaned are connected by means of at least one adapter to a chamber-side end of the air supply, as is performed here by the division of the airflow within the chamber using the at least one adapter, through which the airflow is conducted.

The invention is explained in greater detail hereinafter on the basis of an exemplary embodiment and associated drawings.

### BRIEF DESCRIPTION OF THE DRAWINGS

In the figures:

FIG. 1 shows a schematic structure of the device according to the invention in a side view in an open position

FIG. 2 shows a three-dimensional view of the schematic structure of the device,

FIG. 3 shows a receptacle element for the device,

FIG. 4 shows the device according to FIGS. 1 and 2 in a closed position,

FIG. 5 shows a side view of a device having a first and second adapter within the chamber,

FIG. 6 shows a three-dimensional illustration according to FIG. 6,

FIG. 7 shows an illustration similar to FIG. 6 but without cover in the insert and instead with cross struts 12 between the sides S1 and S2.

### DETAILED DESCRIPTION OF THE PREFERRED EMBODIMENTS

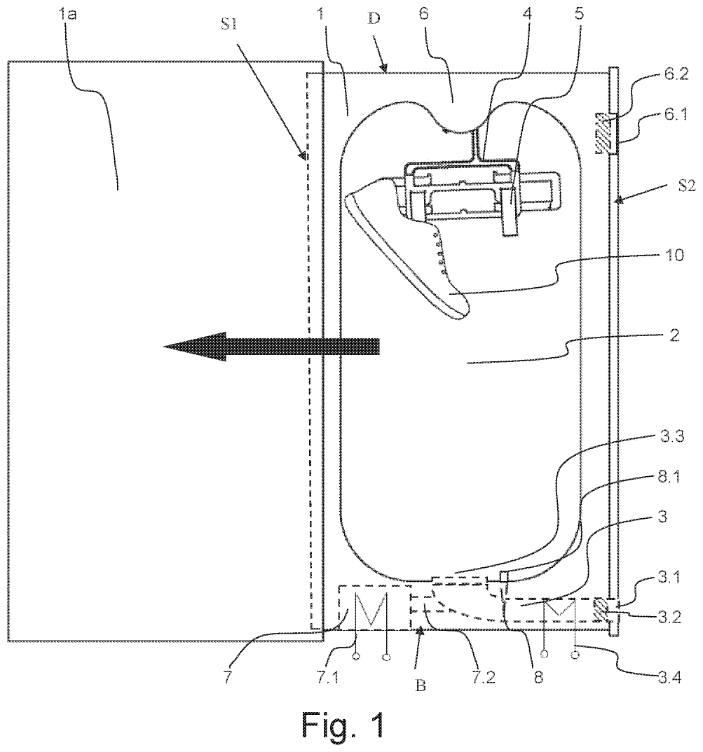

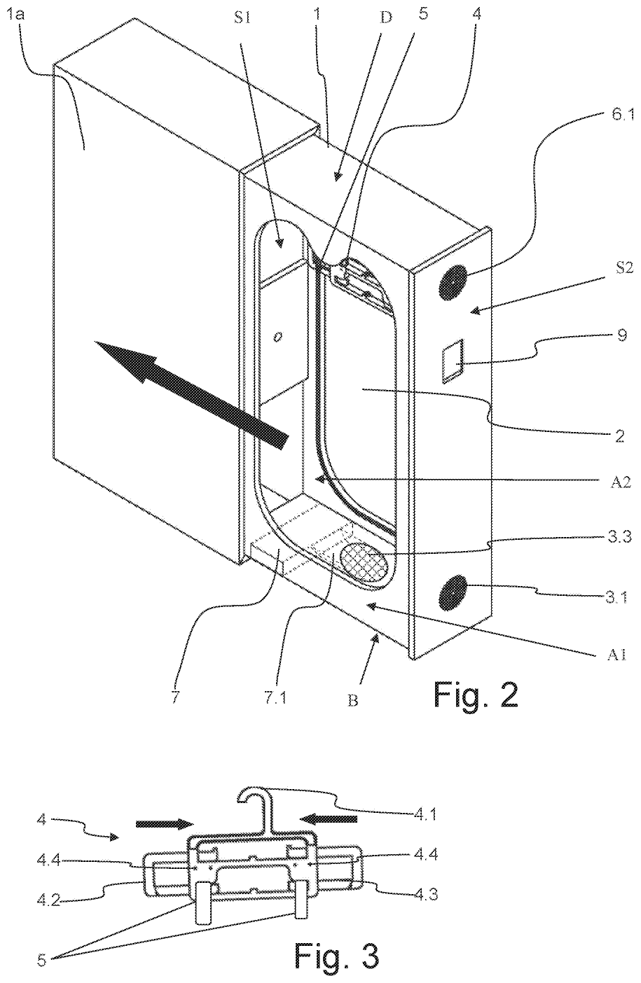

A schematic structure of the device for cleaning and disinfecting textiles, in particular for cleaning and disinfecting the interior of shoes, helmets, gloves, sporting equipment, T-shirts, shirts, suits (pieces of clothing), and bags is shown in FIGS. 1 and 2. The device comprises a housing 1, which forms a chamber 2 in its interior, wherein the chamber 2 can preferably be pushed into and pulled out of a cabinet body 1a in closing the housing 1 via a rail system (not shown). The cabinet body 1a is open in the direction toward the housing 1 (via a vertically extending opening (not identified)), so that the housing 1 can be inserted therein. Otherwise, the cabinet body is closed on the bottom, top, on the sides, and on the rear side. The frame-like housing 1, which can be inserted into the cabinet body 1a, having the chamber element/the chamber 2 is preferably designed like a frame here. The housing 1 includes a bottom B and a lid D, wherein the bottom B and the lid D are connected to one another via two terminal vertical side parts S1, S2 which are parallel to one another. The side part S1 is arranged in the direction toward the cabinet body 1a and the side part S2 is opposite thereto. The side part S2 closes the cabinet body 1a in the inserted state (see FIG. 4). Two covers A1, A2 (see FIG. 1) spaced apart from one another extend between the side parts S1, S2 and the bottom B and the lid D. Each cover A1, A2 has a large passage (not identified), through which objects to be cleaned can be introduced into the chamber 2. The device can thus be loaded from two sides in the open state.

The device 1 includes an air supply 3 in the form of a duct in the lower region, wherein the housing 1 includes on its front an opening in the form of an air inlet 3.1 for the air supply 3 leading into the chamber 2, wherein a fan 3.2 is arranged on the housing-side end of the air supply 3, which forms the air inlet 3.1. The fan 3.2 is designed in such a way that fresh air can be suctioned from the surroundings and conveyed through the air supply 3 into the chamber 2.

The air supply 3 is preferably designed to be tubular and has an angle, preferably of 90°, on its chamber-side end 3.3. The angle acts as a guide of the air, whereby a central outflow of the suctioned-in air into the chamber 2 is achievable.

At least one receptacle element 4, which is arranged hanging on the lid or in the region of the lid of the chamber 2, is provided in the interior of the chamber 2. The receptacle element 4 is designed in the form of a hanger and has a downward facing UV lamp 5 at each end, which generates the UV radiation required for the cleaning.

In an alternative embodiment, the receptacle element is designed in the form of at least one adapter, wherein the receptacle element is connected to the chamber-side end of the air supply and extends into the chamber.

The adapter or the receptacle element includes an air guide, by means of which the air flow guided through the chamber-side end can be divided into a first and second airflow. The adapter has one UV radiation source arranged at an outlet opening of the airflow per airflow.

The outlet openings are arranged radially or axially with respect to the flow of the air depending on the design of the adapter.

The wavelength of the UV radiation is preferably between 100 and 300 nm. The ultraviolet radiation is sufficiently shortwave and high-energy that it is absorbed by molecular oxygen (O₂). The molecular oxygen (O₂) is split into two free oxygen radicals (2 O⁻), which each reacts further with a further molecule of oxygen (O₂) to form ozone (O₃).

The ozone formed in this way at the UV lamp 5 disinfects the textiles of germs, fungi, and bacteria.

Furthermore, the chamber 2 includes an exhaust unit 6 in the lid region of the chamber 2. The exhaust unit 6 is preferably arranged in the upper region of the device, on top in the side part S2 here, and includes, for example, an air outlet 6.1 leading outward through the housing 1 on its front, in which a second fan 6.2 is installed. The fan 6.2 is designed in such a way that the air is suctioned out of the chamber 2, whereby a negative pressure results. An alternating pressure is achieved in the chamber 2, wherein the exhaust unit 6 can be designed in such a way that a vacuum can also be generated in the chamber 2. The vacuum air exchange system removes dead, living, and eliminated organisms from the textiles. The exhaust unit 6 having the air outlet 6.1 can have a replaceable filter (not shown) connected upstream, so that the soiled air can be purified before exiting from the chamber and the ozone can be removed.

For more effective cleaning, the air is heated or cooled or also alternately heated and cooled in the region of the air supply 3. For this method step, a heating unit 3.4 is arranged in the region of the air supply 3, which is preferably wound in the form of a heating coil around the air supply 3. Alternatively, a heating fan can also be used as a combination of heating unit 3.4 and fan 3.2. The air flowing through the air supply 3 is heated in the region of the heating unit 3.4. Alternatively or additionally, a cooling element 6.1 can be provided in the region of the air supply 3.

In one advantageous embodiment, a water tank 7 having a heating element 7.1 arranged therein is provided in the bottom region of the device. The water is heated in such a way that water steam results, wherein the water steam is supplied via a line 7.2 to the air supply 3.

Heated air and the produced water steam are mixed, wherein the mixture is supplied via the chamber-side end 3.3 of the chamber 2.

The piece of clothing to be cleaned is flattened by the air enriched with moisture, whereby later ironing is dispensed with. The water tank 7 is advantageously designed so it can be filled via a lateral opening or can be removed completely.

It is also possible that the bottom B is designed as double-walled and the air is conducted from the air inlet 3.3 via the air supply 3 into the chamber 2 through the bottom-side cavity thus formed. Similarly, the water steam can also flow via the air inlet 3 or a further inlet (not shown) into the chamber 2 through the bottom-side cavity. The fan 3.2, the heating unit 3.4, the water tank 7, and the heating element 7.1 are then also arranged in the bottom-side cavity.

An aroma capsule 8 can preferably be placed in the air supply 3 for producing a fresh odor of the textiles. The released fragrances can be admixed with the air suctioned in by the air supply 3 and conducted into the chamber 2. The odor intensity is preferably settable via a manual regulator 8.1 or via the control 9 using a front-side display of the device.

FIG. 1 furthermore shows the arrangement of a textile to be cleaned, in the form of a schematic shoe 10 here. The shoe 10 is fastened on the receptacle element 4 in such a way that the opening of the shoe extends over the radiation source 5, wherein the radiation source 5 extends into the interior of the shoe 10. The interior of the shoe is thus penetrated by the UV radiation, wherein germs and bacteria are killed by means of the ozone resulting in the shoe 10. Unpleasant odor which is produced by sweat and bacteria can thus be neutralized.

The device moreover offers the option of gently drying damp textiles in a short time.

Environmentally-friendly cleaning and disinfection of the textiles without the supply of chemical cleaning agents is possible using the device according to the invention. Bacteria, germs, and fungi are effectively removed.

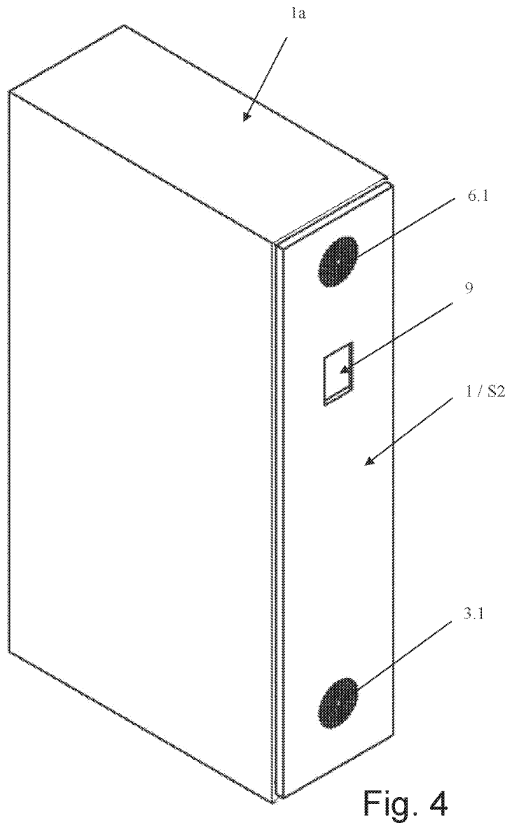

FIG. 3 shows a receptacle element 4 in the form of a hanger having a receptacle 4.1 for hanging the hanger in the chamber. The hanger is adaptable in its width, wherein this is preferably performed by means of carrier elements 4.2, 4.3 movable outward. The carrier elements 4.2, 4.3 are in particular laterally adjustable continuously or by means of detents, whereby the width of the hanger can be set to the different requirements or sizes of the textiles to be cleaned.

The receptacle element 4 furthermore includes connections 4.4 for contacting the UV lamps 5, wherein two UV lamps are used here, for example.

In FIG. 4, the device is shown in the closed state. The insert in the form of the housing 1 is inserted into the cabinet body 1a, wherein the side part S2 terminates the cabinet body 1a. A seal can be provided here between the side part 1 and the cabinet body 1a.

The display 9, the air inlet 3.1, and the air outlet 6.1 are located in the side part S2. Alternatively, the air inlet 3.1 and the air outlet can also be located in the rear side part S1 (not shown), wherein then the cabinet body 1a is to be open at least in these regions on its rear side (not shown).

The device is operated by means of the display 9 or also via an app.

It is possible to hang one or more hangers (receptacles 4) in the chamber 2, so that one or more pieces of clothing can be disinfected and freshened simultaneously.

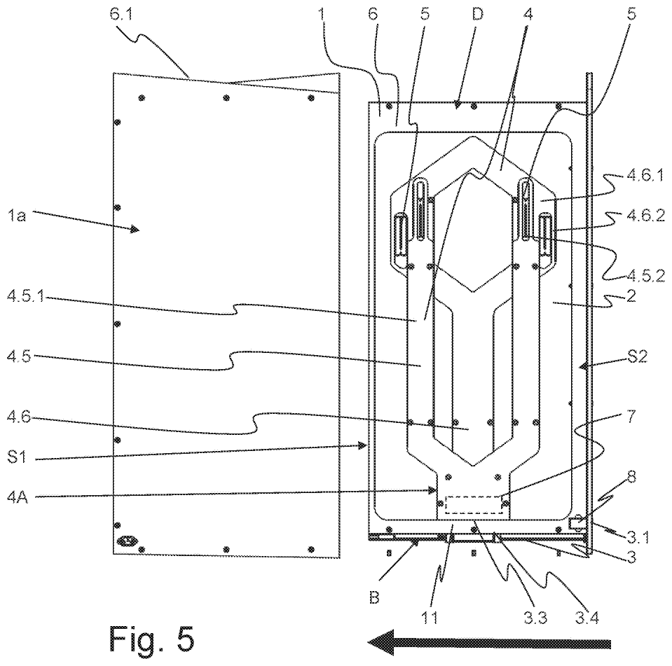

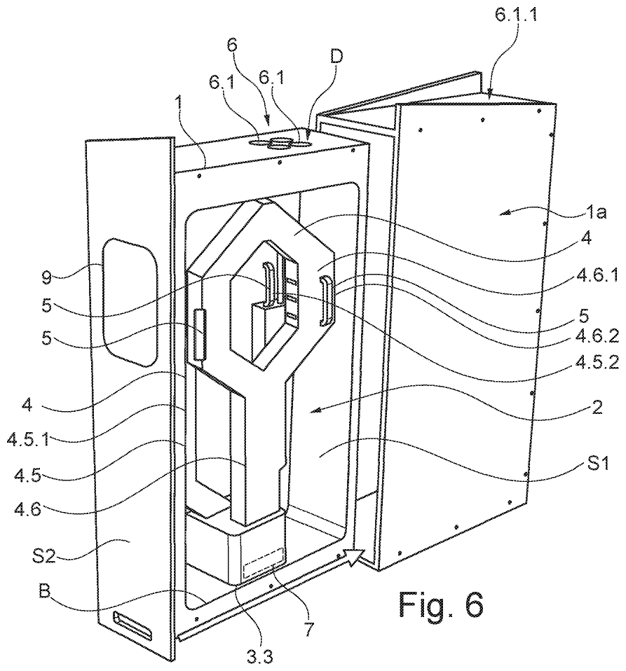

According to FIGS. 5 and 6, the housing 1 includes a chamber 2 having an air supply 3 having an air inlet 3.1 arranged in the front and an alternatively designed receptacle element 4. The receptacle element 4 is designed in the form of a first adapter 4.5 and a second adapter 4.6, which are directly connected to the chamber-side end 3.3 of the air supply 3 and extend upward in the chamber 2. The first and second adapter 4.5, 4.6 are embodied to be hollow and include an air guide 4.5.1, 4.6.1, by means of which the airflow guided through the chamber-side end 3.3 can be divided into a first and second airflow per adapter 4.5, 4.6.

The first and second adapter 4.5, 4.6 include one outlet opening 4.5.2, 4.6.2 per airflow according to FIGS. 5 and 6 here, according to FIG. 5 four outlet openings, in the region of each of which a UV radiation source 5 is arranged. The outlet openings can be arranged radially or axially in relation to the individual airflow. In the present example according to FIG. 5, the first adapter is formed Y-shaped (Y) and includes a first and second UV radiation source 5 on its chamber-side end. Such an adapter is suitable in particular for the positioning of textiles forming a cavity in the form of shoes or helmets, since these can be placed or plugged on one individual UV radiation source 5 or spanning over two UV radiation sources 5.

The second adapter 4.6 differs in its shape from the first adapter 4.5 in such a way that the two airflows or air ducts are brought together again or are connected to one another. The shape of the second adapter 4.6 corresponds to a “Koppa” ( [symbol] in Greek alphabet). The UV radiation sources 5 are arranged in the region of the two separate airflows.

Using such an embodiment of an adapter, textile objects can be hung without a further hanger.

Two adapters are preferably arranged in the chamber 2, wherein two adapters of the first adapter type 4.5, two adapters of the second adapter type 4.6, or one adapter each of the first type 4.5 and the second type 4.6 can be used.

The wiring for the connection of the UV radiation sources 5 is preferably provided in the first and second adapter 4.5, 4.6.

The adapters 4.5, 4.6 can include symmetrically and/or asymmetrically distributed holes and/or passages throughout for distributing the air or the water steam.

A vertically extending region of an air duct 4A is provided above the region of the air supply 3.3 arranged on the chamber side in the bottom B. The first and the second adapter 4.5 and 4.6 adjoin its upper end, which are formed like ducts and by which the airflow is divided further.

It is possible that the adapters 4.5 and 4.6 are formed in one piece with the air duct 4A and are removably and replaceably fastened on the bottom, or that the adapters 4.5 and 4.6 are formed separately and are removably fastened on the upper end of the air duct 4A.

In any case, for example, only one or two or also more first adapters 4.5 or one or two or also more second adapters or a different number and combination of the adapters can also be used, depending on which object is to be cleaned.

According to FIGS. 5 and 6, the water tank 7 is arranged having a heating element arranged therein (not shown) in the region of the transition between the chamber-side end 3.3 of the air supply and the first and second adapter 4.5, 4.6, in the region of the air duct 4A here. The water is heated in such a way that water steam results, wherein the water steam is supplied via a line (not shown) to the air supply 3.

Heated air and the produced water steam are mixed, wherein the mixture is supplied via the chamber-side end 3.3 to the cavities formed in the adapters and is conducted via these to the UV-C radiation sources.

The piece of clothing to be cleaned is smoothed by the air enriched with moisture, whereby later ironing is dispensed with.

The use of a special ironing liquid is also possible, so that the schematically indicated water tank 7 is filled with the ironing liquid instead of water.

The smoothing of the textiles by means of water steam is preferably performed after the UV radiation sources are switched off and thus after the treatment of the textile using the UV radiation.

For more effective cleaning, the air is heated or cooled or also alternately heated and cooled in the region of the air supply 3 as already described above. The heating unit 3.4 is arranged in the region of the air supply 3 for this purpose. Alternatively, a heating fan can also be used as a combination of heating unit 3.4 and fan 3.2. The air flowing through the air supply 3 is heated or cooled in the region of the heating unit 3.4. The temperature is settable and/or changeable in a temperature range of −50° C. to 100° C., preferably in a temperature range of −30° C. to 60° C., particularly preferably in a temperature range between −10° C. and 50° C.

In one advantageous embodiment, an aroma capsule 8 for generating a fresh aroma of the textiles in the air supply 3 is arranged in the region of the air supply 3. The released fragrances can be admixed with the air suctioned in by the air supply 3 and conducted into the chamber 2.

According to the preceding figures, the chamber 2 according to the exemplary embodiment in FIGS. 5 and 6 has an exhaust unit 6 in the lead region of the chamber 2. The exhaust unit 6 is preferably arranged in the upper region of the device and includes an air outlet 6.1 oriented upward and leading outward, in which there is also a fan (not shown). The exhaust unit 6 can also be supplemented with a replaceable filter (not shown), so that the contaminated air can be purified before exit from the chamber and the ozone can be removed. The filter is preferably arranged in the region of the fan and the exhaust unit 6.

An alternating pressure can also be achieved in the chamber 2 using the exhaust unit 6 and air supply 3 according to FIGS. 5 and 6, wherein the exhaust unit 6 can be designed in such a way that a vacuum can also be generated in the chamber 2. The chamber 2 is preferably made airtight for such an embodiment.

It is apparent from FIG. 6 that the exhaust unit 6 can also be arranged having one or more air outlets 6.1 in the lid D. Outlet openings 6.1.1, via which the exhausted air can exit, are then located in the upper side (not shown) of the cabinet body 1a. In the inserted state of the housing 1, the outlet openings 6.1.1 of the cabinet body 1a are located above the air outlets 6.1 in the lid D of the housing 1.

Furthermore, an additional pressure system 11 is possible in the region of the bottom B of the chamber 2, by means of which the air supply 3 and the exhaust unit 6 for generating a negative pressure or overpressure are assisted.

The pressure in the chamber 2 can be settable and/or changeable in a pressure range between 0.001 bar to 10 bar, in particular between 0.1 bar and 2 bar.

The use of a filter is particularly recommended since escaping ozone is harmful to health due to irritation of the respiratory tract even at low concentrations.

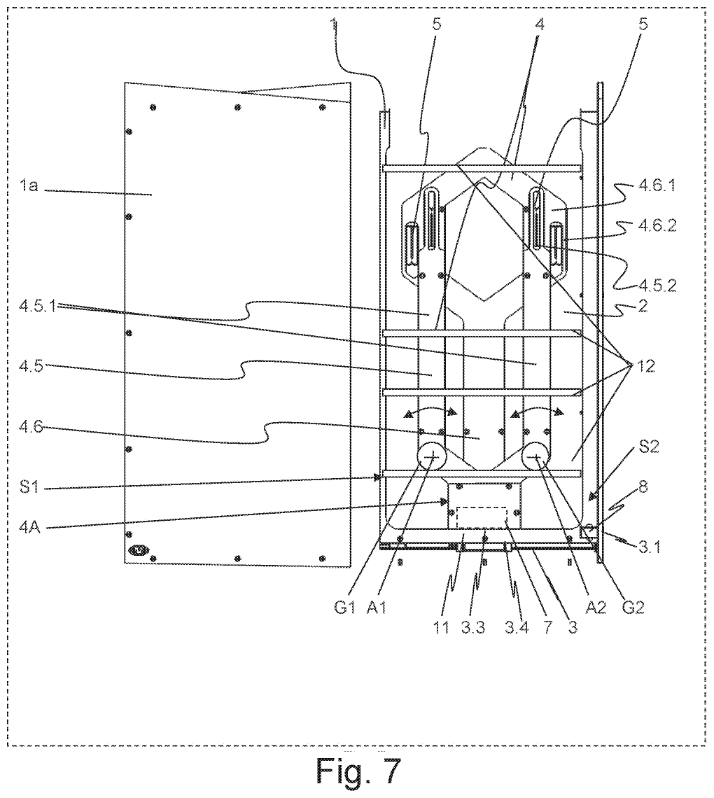

FIG. 7 shows an illustration, similar to FIG. 6 but without lid in the insert and instead having cross struts 12 between the sides S1 and S2, which stabilize the sides S1, S2 in relation to one another and ensure that the objects to be cleaned do not protrude laterally out of the housing 1 and prevent the insertion into the body. It is possible here to insert the objects to be cleaned from above. The exhaust (not shown here) is then arranged as in FIG. 1 in the front side part S2 or also in the rear side part S2, wherein then corresponding air outlets are provided in the cabinet body 1a in the rear wall. It is indicated in this variant that the first and the second adapter 4.5 and 4.6 are provided detachably adjoining the air duct 4A.

Instead of cross struts, one or more platelike elements can also extend between the side parts S1, S2.

Such cross struts or platelike elements can also be used in the above-described variants.

Furthermore, it is possible according to FIG. 7 that, for example, the first adapter 4.5 includes a joint G1 and G2 in each of the two upwardly extending regions of the air guide 4.5.1. The region shown here extending vertically upward in a starting position, for example, is thus designed to be pivotable around a horizontal axis A1, A2, which is indicated by the double arrows. The upper ends of the two air guides 4.5.1 of the adapter 5.5 can thus be pivoted toward one another and away from one another and the distance thereof can thus be adapted to the objects to be received.

Using the device according to the invention, the airflow supplied to the chamber is divided via the ducts which are formed by the adapter elements in the chamber and guided in each case to one UV radiation source 5.

The number of the ducts formed in adapter correspond here to the number of the UV radiation sources 5 provided on the adapter.

It is thus also possible to provide an adapter not only with two, but also with three, four, or more upwardly extending branches, which each guide the airflow to one UV radiation source 5.

The UV radiation source 5 is preferably located at the air exit regions of the adapter.

The adapters also form the receptacles for the objects to be cleaned.

The adapters can be embodied here so that the distance of the upper regions which receive the objects to be cleaned is changeable.

It is possible to arrange multiple of the essentially cuboid devices according to the invention adjacent to one another and/or one over another like batteries. The devices can also be connected to one another in this case.

It is furthermore also alternatively possible to provide a cabinet body having a pivotable or laterally displaceable door, wherein the chamber/cleaning chamber is then formed in the cabinet body.

Depending on the textile to be cleaned, the individual parameters such as pressure, temperature, UV radiation, and ozone can be set. Using the maximum power of the device is advisable, for example, in the cleaning of leather, while cashmere is only to be treated carefully, for example.

### LIST OF REFERENCE NUMERALS

1 housing

2 chamber

3 air supply

3.1 air inlet

3.2 fan

3.3 chamber-side end of the air supply

3.4 heating element

4 receptacle element

4.1 receptacle

4.2 carrier element

4.3 carrier element

4.4 connection

4.5 first adapter

4.5.1 air guide

4.5.2 outlet opening

4.6 second adapter

4.6.1 air guide

4.6.2 outlet opening

4A vertically extending region

5 UV radiation source

6 exhaust unit

6.1 air outlet

6.1.1 outlet openings

6.2 fan

7 water tank

7.1 heating element

7.2 line

8 aroma capsule

8.1 manual regulator

9 control having display

10 shoe

11 pressure system

12 cross struts

A1, A2 covers

B bottom

D lid

S1, S2 side parts

1. A method for cleaning and disinfecting at least one object, in particular an object including a textile, wherein the object is inserted through an opening into a chamber enclosed by a housing, characterized in that the object is received in the chamber by means of a receptacle element and at least three of the following techniques a) to d) are implemented during a cleaning cycle in the chamber:
a) UV-C light in the nonvisible wavelength range is generated by means of at least one UV lamp,
b) ozone is generated by at least one means for ozone generation,
c) the temperature modes are settable and/or selectable,
d) the pressure is settable and/or changeable,
wherein by means of an air inlet leading from the housing into the chamber and an air outlet leading from the chamber out of the housing, an airflow is produced inside the chamber.

2. The method according to claim 1, characterized in that the means for ozone generation is the UV lamp, wherein the UV lamp is designed to be dimmable.

3. The method according to claim 1, characterized in that the temperature modes are settable or changeable in such a way that the temperature rises in the chamber from a low temperature to a higher temperature, or is reduced from a high temperature to a lower temperature, or the temperature alternately rises and sinks again or sinks and rises again.

4. The method according to claim 3, characterized in that the temperature is settable and/or changeable in a temperature range from −30° C. to 150° C.

5. The method according to claim 1, characterized in that a negative pressure and/or an overpressure can be generated in the chamber in relation to the atmospheric pressure, and/or the pressure in the chamber rises from a low pressure to a higher pressure or is reduced from a high pressure to a low pressure, or the pressure alternately rises and sinks again or sinks and rises again.

6. The method according to claim 5, characterized in that the pressure is settable and/or changeable in a pressure range from vacuum to 5 bar.

7. The method according to claim 1, characterized in that silver nanoparticles having a particle size from 25 nm to 1000 nm are introduced from a reservoir into the chamber.

8. The method according to claim 1, characterized in that heated or cooled air is introduced into the chamber and is irradiated in the chamber using ultraviolet light from at least one UV-C light source, and ozone is generated in the chamber by means of the UV-C light source and thus disinfection and cleaning of the at least one object takes place, wherein the UV-C light source is at least partially introduced into the recesses or the cavity of the object, and an alternating pressure is generated in the chamber by means of an exhaust unit, whereby soiling of the object is released and dirt particles are transported away.

9. The method according to claim 1, characterized in that the airflow is heated by means of a heating element in the region of the suctioning.

10. The method according to claim 1, characterized in that water steam is admixed with the airflow in the region of the supply into the chamber in such a way that the object to be cleaned is smoothed.

11. The method according to claim 10, characterized in that the water steam is produced in a water tank having integrated heating element.

12. The method according to claim 1, characterized in that an aroma is supplied to the airflow and/or the chamber.

13. A device for cleaning and disinfecting at least one object, wherein the device includes a chamber enclosed by a housing, in which the at least one object is received on a receptacle element, characterized in that the device includes at least three of the following means A) to D):
A) at least one UV lamp arranged in the chamber on the receptacle element for generating UV-C light in the nonvisible wavelength range,
B) at least one means for ozone generation,
C) at least one unit for setting and/or changing the temperature in the chamber,
D) at least one unit for setting and/or changing the pressure in the chamber,
an air supply pointing from outside the housing into the interior of the chamber having an air inlet and at least one air outlet leading from the chamber out of the housing.

14. The device according to claim 13, characterized in that the receptacle element is designed in the form of a hanger and includes at least one UV lamp for generating UV-C radiation and for generating ozone, and the device includes an exhaust unit on the air outlet for generating a negative pressure in the chamber.

15. The device according to claim 13, characterized in that the receptacle element includes two UV lamps spaced apart from one another and arranged adjacent to one another.

16. The device according to claim 13, characterized in that the receptacle element is adjustable in its width.

17. The device according to claim 13, characterized in that the receptacle element is designed in the form of at least one adapter, and the receptacle element is connected to the chamber-side end of the air supply and extends into the chamber 2.

18. The device according to claim 17, characterized in that the adapter includes an air guide, by means of which the airflow guided through the chamber-side end can be divided into multiple airflows, and the adapter includes one UV radiation source arranged on an outlet opening of the airflow per airflow.

19. The device according to claim 18, characterized in that the outlet openings are arranged radially or axially in relation to the flow of the air.

20. The device according to claim 3, characterized in that the receptacle element is replaceable, and/or multiple receptacle elements are arrangeable inside one chamber.

21. The device according to claim 13, characterized in that the cleaning cycle is settable by means of a control unit and the parameters such as temperature and/or pressure and/or addition of fragrances and/or silver nanoparticles are settable.

22. The device according to claim 13, characterized in that an aroma element is arranged in the air supply.

23. The device according to claim 13, characterized in that the device includes a water tank having a heating element in such a way that water steam can be produced, and the water steam can be supplied via a duct to the air supply.

24. The device according to claim 13, characterized in that the chamber can be pulled out of the housing by means of a rail system.

25. The device according to claim 1, characterized in that the receptacle for the objects is variable in its width.
